(19) United States
(12) Patent Application Publication
(10) Pub. No.: US 2024/0156533 A1
(43) Pub. Date: May 16, 2024

(54) ROBOTIC COLD ATMOSPHERIC PLASMA SURGICAL SYSTEM AND METHOD

(71) Applicant: US Patent Innovations, LLC, Takoma Park, MD (US)

(72) Inventors: Jerome Canady, Lakeland, FL (US); Changhan Jun, Beltsville, MD (US); Taisen Zhuang, Rockville, MD (US)

(21) Appl. No.: 18/280,062

(22) PCT Filed: Mar. 4, 2022
(86) PCT No.: PCT/US22/18932

§ 371 Date: Sep. 1, 2023

Related U.S. Application Data
(60) Provisional application No. 63/301,282, filed on Jan. 20, 2022.
(60) Provisional application No. 63/156,787, filed on Mar. 4, 2021.

(51) Int. Cl. A61B 34/10; A61B 34/20; A61B 34/30; A61B 34/37; A61B 90/00

(52) U.S. Cl. CPC A61B 34/10; A61B 34/20; A61B 34/37; A61B 90/361; A61B 2034/107; A61B 2034/2059; A61B 2034/2074; A61B 2034/305; A61B 2560/0437

(57) ABSTRACT

A system and method in accordance with the present invention controls the dosage of cold plasma generated multi-species delivered to a patient, the distance of the CAP probe should keep constant around 1.5-2.5 mm as well as the treating time and treating area should be controlled during the procedure. The robotic system will have a quick attachable connection to the CAP probe and the robotic system will keep the constant distance from CAP tip's end to patient body, at same time provide surface scan with computer planned controllable surface treatment area, treating time and step distance between two return scans.

### CROSS-REFERENCE TO RELATED APPLICATIONS

The present application claims the benefit of the filing date of U.S. Provisional Patent Application Ser. No. 63/301,282 filed by the present inventors on Jan. 20, 2022, and U.S. Provisional Patent Application Ser. No. 63/156,787 filed by the present inventors on Mar. 4, 2021.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

### Field of the Invention

The present invention relates to robotic surgical systems, and more specifically, a robotic surgical navigation system.

### Brief Description of the Related Art

Within the scope of minimally-invasive surgery, such as endoscopic or laparoscopic surgery, access to the operating site is made via small incisions in the body of the patient (such as the abdomen or thorax), in which the practitioner places a cannula formed by a tube whereof the diameter typically varies from 3 to 15 mm, through which the practitioner can insert into the body of the patient either an endoscope for obtaining a video image on a monitor, or long and fine instruments for performing a procedure at the operating site.

Manipulation of an intervention device through small incisions or through a natural orifice requires in both cases to move it around a fixed point or center of motion, which corresponds to the incision or natural orifice itself. Such incision or natural orifice is herein generally referred as a point of penetration in the patient.

Since the surgeon generally has both hands occupied by the surgical instruments, an assistant is necessary to maintain any other intervention device in a desired position, in particular the endoscope that is used to guide the surgeon in his surgery.

Robotic systems have been developed to handle and displace the endoscope in the place of the assistant. Examples of modern systems include those disclosed in U.S. Pat. No. 10,639,066 entitled “System for Controlling Displacement of an Intervention Device,” which discloses a system for controlling displacement of an intervention device having an end for inserting in a patient body, including a base in a fixed position relative to the patient. A first portion has an arc member and is pivotally mounted on the base around a first axis (A1). A second portion includes a support member and a carrier member. The support member partially rotates around a second axis (A2). A third portion includes a holding member, and a sliding member mounted on the support member along a translation axis (A.sub.T). The holding member is arranged so that translation of the sliding member causes the intervention device to translate along a third axis (A3). The third axis (A3) is parallel to and offset from the translation axis (A.sub.T). When the carrier member is positioned halfway of the arc member, the first (A1), second (A2) and third (A3) axes are orthogonal.

Similarly, a variety of systems have been proposed for providing gas-assisted electrosurgery. U.S. Pat. No. 4,429,694 to McGreevy disclosed an electrosurgical generator and argon plasma system and a variety of different electrosurgical effects that can be achieved depending primarily on the characteristics of the electrical energy delivered from the electrosurgical generator. The electrosurgical effects included pure cutting effect, a combined cutting and hemostasis effect, a fulguration effect, and a desiccation effect. Fulguration and desiccation sometimes are referred to collectively as coagulation.

Another method of monopolar electrosurgery via argon plasma technology was described by Morrison in U.S. Pat. No. 4,040,426 in 1977 and McGreevy U.S. Pat. No. 4,781,175. This method, referred to as argon plasma coagulation (APC) or argon beam coagulation is a non-contact monopolar thermoablative method of electrocoagulation that has been widely used in surgery for the last twenty years. In general, APC involves supplying an ionizable gas such as argon past the active electrode to target tissue and conducting electrical energy to the target tissue in ionized pathways as non-arcing diffuse current. Canady described in U.S. Pat. No. 5,207,675 the development of APC via a flexible catheter that allowed the use of APC in endoscopy. These new methods allowed the surgeon, endoscopist to combine standard monopolar electrocautery with a plasma gas for coagulation of tissue.

Yet another system is disclosed in U.S. Patent Application Publication No. 2013/0296846, which disclosed a system for simultaneously cutting and coagulating tissue. Another system, referred to as a “cold atmospheric plasma” system, is disclosed in U.S. Patent Application Publication No. 2014/0378892.

Several different systems and methods for performing Cold Atmospheric Plasma (CAP) treatment have been disclosed. For example, U.S. Published Patent Application No. 2014/0378892 discloses a two-electrode system for CAP treatment. U.S. Pat. No. 9,999,462 discloses a converter unit for using a traditional electrosurgical system with a single electrode CAP accessory to perform CAP treatment.

### SUMMARY OF THE INVENTION

In a preferred embodiment, the present invention is a system and method for controlling a dosage of cold plasma generated multi-species delivered to a patient. The distance of the CAP probe should be kept constant about 1.5-2.5 mm as well as the treating time and treating area should be controlled during the procedure. A robotic system such as is disclosed in U.S. Pat. No. 10,639,066 will have a quick attachable connection to the CAP probe, and the robotic system will keep a constant distance from CAP probe tip's end to target tissue and at same time provide a surface scan with computer planned controllable surface treatment area, treating time and step distance between two return scans.

In a preferred embodiment, the present invention is a method for robotic controlled cold atmospheric plasma surgery. The method comprises defining a tissue area to be treated, inputting cold atmospheric plasma settings into a cold atmospheric plasma surgical system, calculating with a processor a robot movement path to cover the defined tissue area at the inputted cold atmospheric plasma settings and storing the calculated path in memory, simulating with the calculated robot movement path, and activating a cold atmospheric plasma robot movement plan, wherein the cold atmospheric plasma robot movement plan comprises robotically moving a cold atmospheric plasma probe connected to the robotic arm end-effector in accordance with the calculated robot movement path and applying cold atmospheric plasma to the defined tissue area to be treated at the inputted cold atmospheric plasma settings. The defining step comprises detecting at least three points inputted by a user to define an area to be treated. The step of detecting at least three points inputted by a user to define an area to be treated may comprise detecting a manual force applied to a robotic arm end-effector with at least one sensor, converting the detected manual force into robot instructions with a processor, moving the robotic arm with the processor in accordance with the instructions, and recording the movement path.

In another preferred embodiment, the present invention is a system for robotic controlled cold atmospheric plasma surgery comprising a passive positioning system having a positioning system base, at least one passive positioning arm extending from the positioning system base, the passive positioning arm having five degrees of freedom, a robotic end effector connected to the passive positioning arm, the robotic end effector having three degrees of freedom and a connector for connecting a cold atmospheric plasma accessory to the robotic end effector, a master control module in the base for controlling robotic movement of the robotic end effector, and a cold atmospheric plasma generator.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is a flow chart illustrating a method of a first preferred embodiment of the present invention.

FIG. 2 is a side perspective view of a robot end-effector and cold plasma accessory in accordance with a preferred embodiment of the present invention.

FIG. 3 is a diagram illustrating performance of the method of the first preferred embodiment of the present invention.

FIG. 4 is a flow chart illustrating a method of a second preferred embodiment of the present invention.

FIG. 5 is a diagram illustrating performance of the method of the second preferred embodiment of the present invention.

FIG. 6 is a system diagram of a system in accordance with a preferred embodiment of the present invention.

FIG. 7 is a perspective view of an embodiment of the present invention including a surgical robotic positioning arm in a surgical setting.

FIG. 8 is a perspective view of surgical robotic positioning arm in accordance with a preferred embodiment of the present invention.

FIGS. 9A, 9B and 9C are side views illustrating operation of a surgical robotic positioning arm in accordance with a preferred embodiment of the present invention.

FIG. 10 is a side perspective view illustrating operation of a surgical robotic positioning arm in accordance with a preferred embodiment of the present invention.

FIGS. 11A and 11B illustrate an embodiment of the present invention including a plurality of surgical robotic positioning arms.

### DETAILED DESCRIPTION OF THE PREFERRED EMBODIMENTS

A preferred embodiment of the present invention is described with reference to the drawings.

A system and method in accordance with the present invention controls the dosage of cold plasma generated multi-species delivered to a patient. The distance of the CAP probe should be kept constant in a range of 1.5-2.5 mm. The treatment time and treatment area should be controlled during the procedure. The robotic system will have a quick attachable connection of the CAP accessory to the robotic arm end-effector, and the robotic system will keep the constant distance range from the CAP accessory tip's end to the target tissue, at same time provide surface scan with computer planned controllable surface treatment area, treating time and step distance between two return scans.

With the example of a robotic system voice-controlled laparoscope robotic arming system such as is disclosed in U.S. Pat. No. 10,639,066, the new system and method can control the CAP probe to provide automatic cold plasma cancer treatment with the following features:
surface measure/detection,
distance measure/detection,
route planning,
Communication with Canady Helios Cold Plasma Generator
Emergency detection and solutions
Multi-display data presentation
GUI human interface
Human language command/speech interface
Motor Control and power control
Image detection and processing.

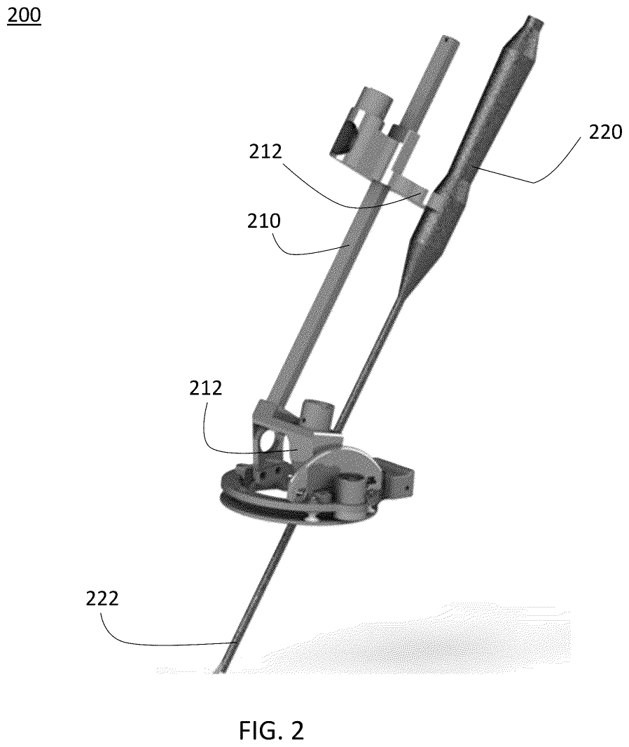

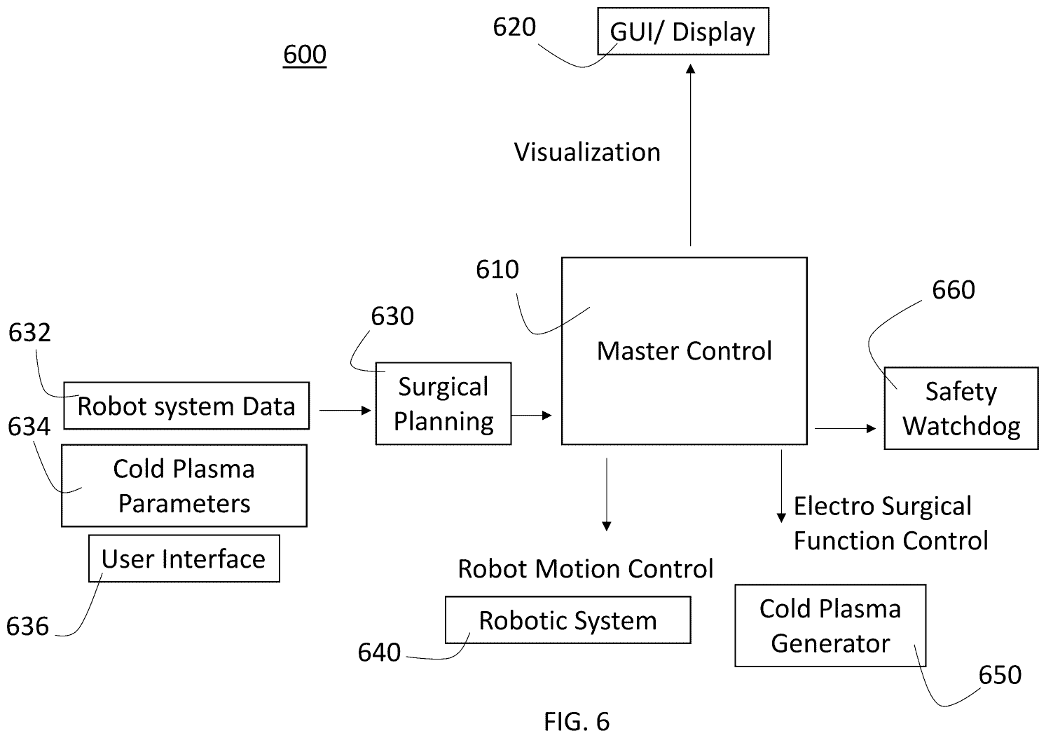

The robotic system has three degrees of freedom: 2 rotational and 1 translational. An exemplary embodiment 200 of a robotic arm end-effector 210 having a connector or connectors 212 for connecting cold atmospheric plasma (CAP) accessory or probe 220 to the end effector is shown in FIG. 2. As shown in FIG. 6, the system 600 has a mater control 610, a graphical user interface (GUI) and display 620, a surgical planning subsystem 630 having a robotic system data 632, cold atmospheric plasma (CAP) data 634, user interface 636, robotic system 640, cold atmospheric plasma generator 650 and safety watchdog 660. The under interface 636 may be the GUI 620 or may be several inputs: voice command, handheld force drive: robot follows the users input at constant speed, foot-pedal, autonomous trajectory tracking based on motion planning, and force sensing at the tool tip not to damage the tissue during the robot motion. The GUI/display 620 may be part of the cold plasma generator 650, part of the robotic system 640 or independent. The mater control 610 similarly may be part of the cold plasma generator 650, the robotic system 640 or may be a separate unit. The robotic system data 632 and cold plasma parameters 634 may be stored in memory or storage in the master control 610 or elsewhere.

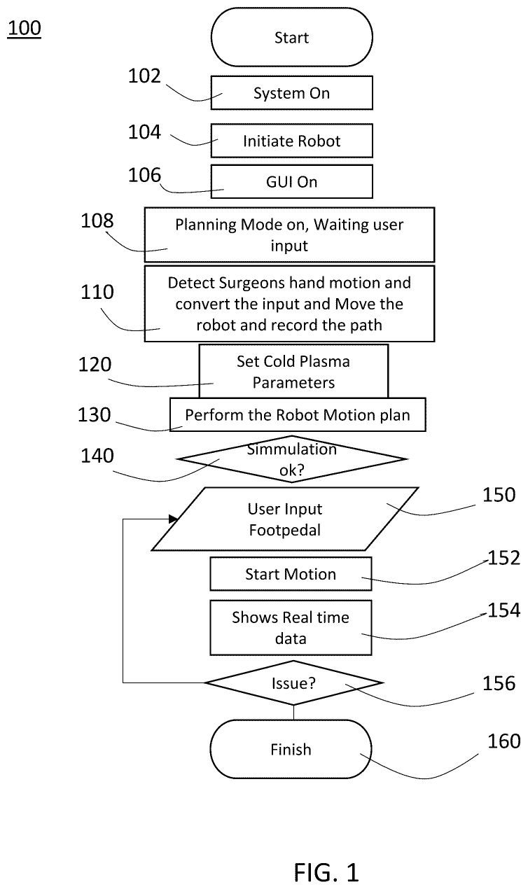

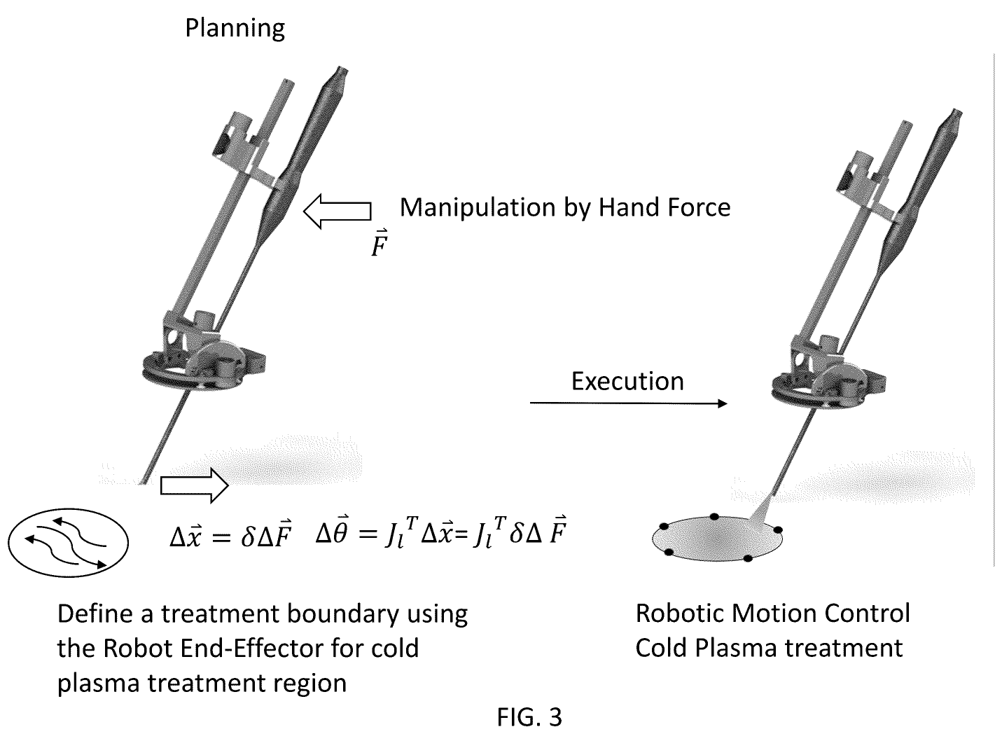

Two methods are available for surgical planning. In the first method, which is shown in FIGS. 1 and 3, the user turns the system on 102. The surgical robot or robotic arm(s) is initiated 104. The graphical user interface also is turned on or initiated 106. The robotic system 600 then entrees planning mode 108 in which it waits for user input. The user then enters the boundaries of the area to be treated 110 and enters the CAP parameters 120. The user may enter the boundaries of the area to be treated in a variety of ways. First, the system may employ a motion sensing system with which the surgeon manipulates the end-effector by holding and moving it to identify three or more points defining the area to be treated. In one embodiment, the robot motion sensing will sense the hand force applied to the end-effector and then move the end effector in accordance with that force. In this manner, the user draws a boundary of the resection region to be treated. The master control stores the data defining the boundary of the area to be treated. The “sensing” of the movement may be, for example, the robot motor having a counter the number of steps in each direction of movement. The “sensor” further could be a 3D sensor or a camera. In another embodiment, the user may move the end-effector, for example, using a joystick control. In yet another embodiment, a camera can be used to track the movement of the end effector by the user.

Once the boundaries of the area to be treated are defined, the master control, e.g., a processor in the master control, calculates a path to cover the area within the boundary with CAP parameters set by the surgeon (treatment time, speed, repeats . . . ). The robot then treats the patient with cold plasma using the robot's motion control system. Once the boundary has been defined and the path or motion plan has been calculated by the master control, the robot performs the motion plan without activating the CAP system to effectively simulate the motion plan 130. If the simulation is approved 140, the user activates the cold plasma 150, for example, using a foot pedal, and the robot starts motion 152 of the end-effector to perform the motion plan. Real-time data may be displayed 154, for example, on the GUI/display. If the treatment is deemed effective the method is finished 160 and the system is deactivated. If the treatment is deemed ineffective 156, the user may re-initiate the motion plan.

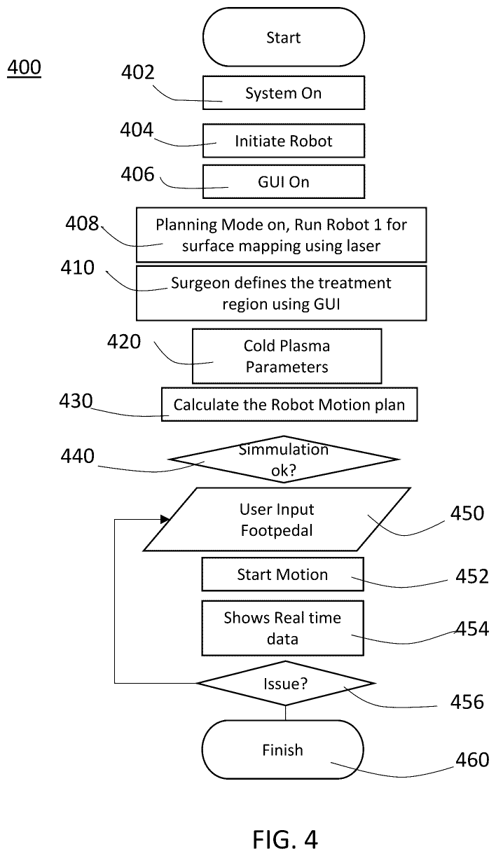

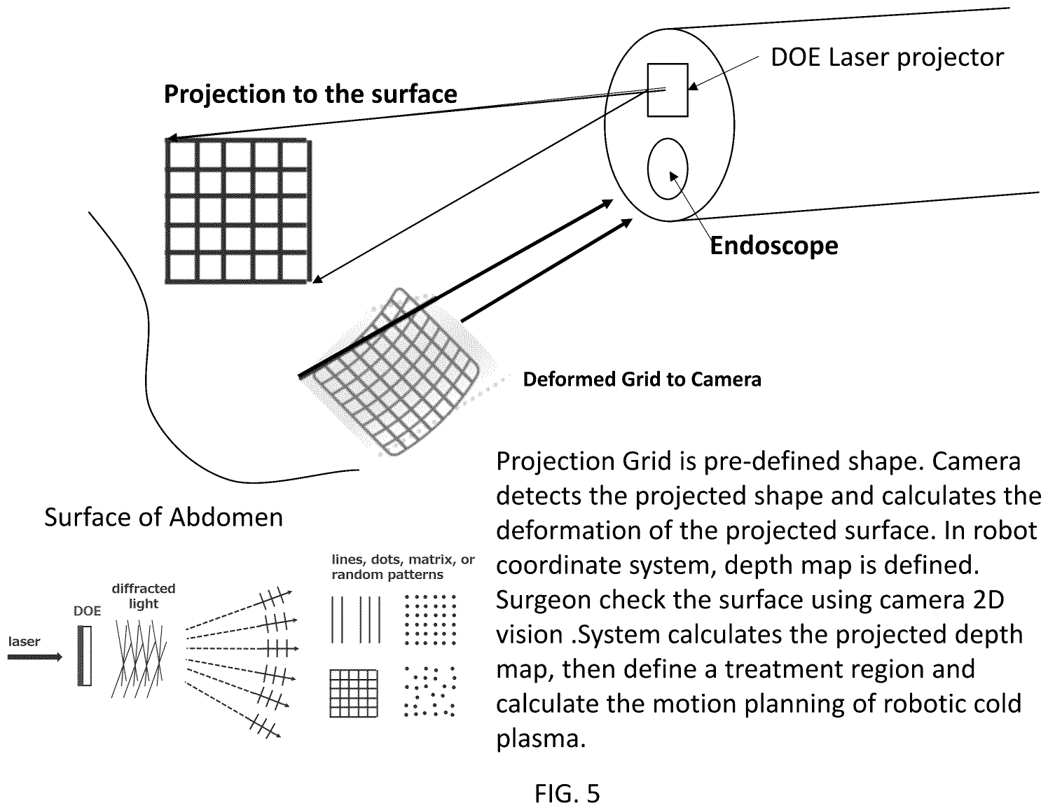

In the second method, which is shown in FIGS. 3-5, two robots (Robot 1 and Robot 2) are co-registered to the base coordinate system using robot arm base information and IMU sensing of two systems. The endoscopic camera and the Camera with a laser DOE (diffractive optical element) projector are mounted to Robot 1. The camera position is calibrated to the robot base coordinate system.

The user turns the system on 402. The surgical robotic arms are initiated 404. The graphical user interface also is turned on or initiated 406. The robotic system 600 then enters planning mode 408 in which it waits for user input. The user then enters the boundaries of the area to be treated 410 and enters the CAP parameters 420. A projection grid is a shape defined by the user. The camera detects the projected shape, and the processor calculates the deformation of the projected surface. In the robot coordinate system, a depth map is defined.

Once the boundaries of the area to be treated are defined, the master control, e.g., a processor in the master control, calculates a path to cover the area within the boundary with CAP parameters set by the surgeon (treatment time, speed, repeats . . . ). Once the boundary has been defined and the path or motion plan has been calculated by the master control, the robot performs the motion plan without activating the CAP system to effectively simulate the motion plan 430. The surgeon checks the surface of the area to be treated using camera 2D vision. If the simulation is approved 440, the user activates the cold plasma 450, for example, using a foot pedal, and the robot starts motion 452 of the end-effector to perform the motion plan. The system calculates the projected depth map, then defines a treatment region. The robot then calculates the path to cover the boundary with setting parameters (treatment time, speed, repeats . . . ) relative to the Robot 2 coordinate system. Real-time data may be displayed 454, for example, on the GUI/display. If the treatment is deemed effective the method is finished 460 and the system is deactivated. If the treatment is deemed ineffective 456, the user may re-initiate the motion plan.

Trajectory Tracking with Cold Plasma

The robot will execute the motion of the cold plasma probe. The system requires the surgeon to activate the foot pedal to execute the motion for safety purpose. Robot motion and cold plasma function will be active only when the surgeon holds the foot pedal.

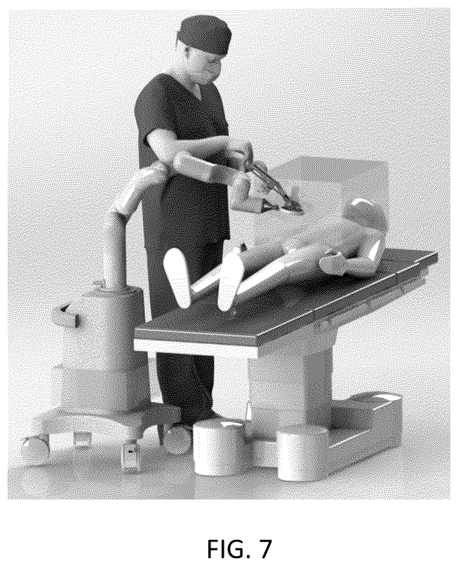

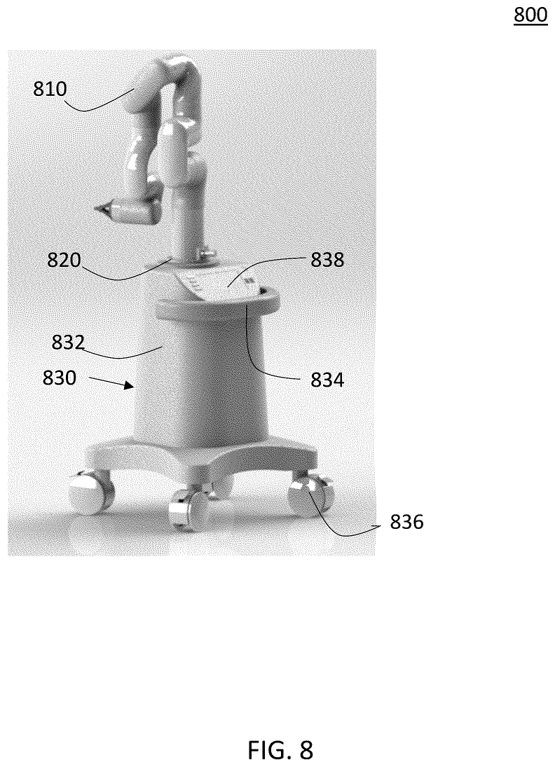

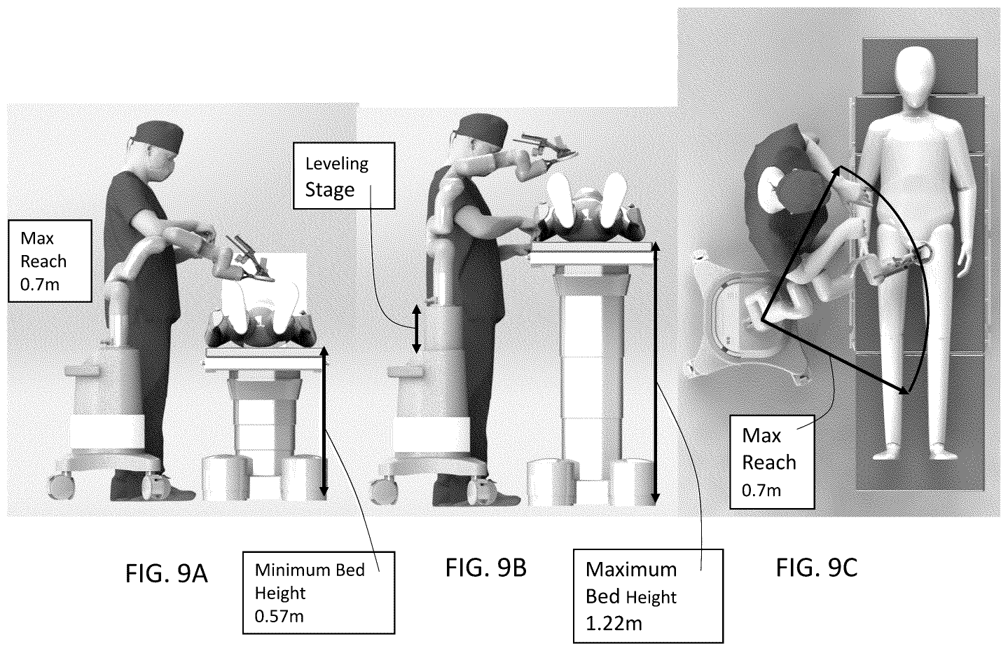

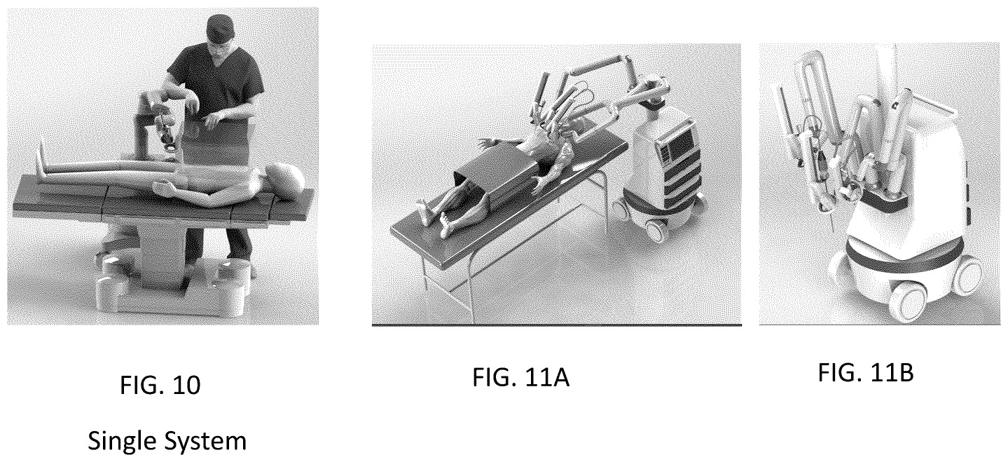

The robot further may include one or a plurality of surgical positioning arms as shown in FIGS. 7-11B. Each surgical positioning arm has 5 degrees of freedom, a 5 kg payload, and a maximum reach of at approximately 700 mm. The robotic positioning arm holds the robot end-effector at the target position. Since the arm is passive, it holds no risk to the patient and has no risk of mechanical failure. The positioning system 800, shown in FIGS. 8-11B, has a modular design to allow combining of up to three positioning arms in one system. The positioning system 800 has three major components: a positioning arm 810, a leveling stage 820 and abase platform or cart 830. The positioning system levelling stage 820 or cart 830 may hold some or all the modules shown in FIG. 6, such as the master control, the robot control, and the cold plasma generator. The positioning system cart 830 has a main body 832, a push bar 834, and a plurality of caster wheels 836 with a position locking mechanism such as locking wheels, outriggers, or hydraulic support. A user interface 840 in the positioning system may have touch display (GUI), physical controls or buttons, status indicators such as LED's, a power switch, stop button, voice control and audio feedback. FIGS. 11A and 11B shows a position system having a plurality of positioning arms, e.g., three arms.

The surgical robotic position arm or fixture has the following features:
Position& Hold the robotic system.
Quick and Precise Fixture
Collaboration up to 3 platforms

The Robot Arm is used as a passive mechanical fixture to locate the end-effector for laparoscopic surgery, so that the RCM of the end-effector is placed at the entry of the port on the patient body surface. A user can hold the wrist of the robot arm, then the robot arm senses the user's intension by reading force and torque at the robot arm wrist, manipulates the arm to follow the user's motion. Once the user places the end-effector at the designated position, the motion of the robot arm and all joints of the arm must be locked at the position.

The foregoing description of the preferred embodiment of the invention has been presented for purposes of illustration and description. It is not intended to be exhaustive or to limit the invention to the precise form disclosed, and modifications and variations are possible considering the above teachings or may be acquired from practice of the invention. The embodiment was chosen and described to explain the principles of the invention and its practical application to enable one skilled in the art to utilize the invention in various embodiments as are suited to the particular use contemplated. It is intended that the scope of the invention be defined by the claims appended hereto and their equivalents. The entirety of each of the aforementioned documents is incorporated by reference herein.

1. A method for robotic controlled cold atmospheric plasma surgery comprising:
defining a tissue area to be treated, said defining comprising:
inputting cold atmospheric plasma settings into a cold atmospheric plasma surgical system;
calculating with a processor a robot movement plan to move a cold atmospheric plasma probe over the defined tissue area at said inputted cold atmospheric plasma settings and storing said calculated plan in memory, wherein said cold atmospheric plasma robot movement plan comprises robotically moving a cold atmospheric plasma probe connected to a robotic arm end-effector over the defined tissue area in accordance with the calculated robot movement path;
robotically moving said cold atmospheric plasma probe over said defined tissue area according to said calculated robot movement plan without activating cold atmospheric plasma; and
activating said cold atmospheric plasma robot movement plan to move said cold atmospheric plasma probe over said defined tissue area according to said calculated robot movement plan while activating cold atmospheric plasma to treat the defined tissue area with cold atmospheric plasma.

2. A method for robotic controlled cold atmospheric plasma surgery according to claim 1, wherein said step of defining a tissue area to be treated comprises detecting at least three points inputted by a user to define an area to be treated. and said step of detecting at least three points comprises:
detecting a manual force applied to a robotic arm end-effector with at least one sensor;
converting the detected manual force into robot instructions with a processor;
moving the robotic arm with said processor in accordance with the instructions; and
recording the movement path.

3. A system for robotic controlled cold atmospheric plasma surgery comprising:
a passive positioning system comprising:
a positioning system base;
at least one passive positioning arm extending from said positioning system base, said passive positioning arm having five degrees of freedom;
a robotic end effector connected to said passive positioning arm, said robotic end effector having three degrees of freedom and a connector for connecting a cold atmospheric plasma accessory to said robotic end effector;
a master control module in said base for controlling robotic movement of said robotic end effector; and
a cold atmospheric plasma generator.

4. A system for robotic controlled cold atmospheric plasma surgery according to claim 3 wherein said passive positioning system further comprises a levelling stage.

5. A method for robotic controlled cold atmospheric plasma surgery according to claim 1, wherein said movement plan comprises maintaining a constant distance between a tip end of said cold atmospheric plasma probe and the tissue.

6. A method for robotic controlled cold atmospheric plasma surgery according to claim 1, wherein said movement plan comprises moving said cold atmospheric plasma probe at a constant speed.

7. A method for robotic controlled cold atmospheric plasma surgery according to claim 5, wherein said constant distance between said tip end of said cold atmospheric plasma probe and the tissue is in the range of 1.5-2.5 mm.

8. A system for robotic controlled cold atmospheric plasma surgery according to claim 3 wherein said three degrees of freedom of said robotic end effector comprise two rotational degrees of freedom and one translational degree of freedom.

9. A method for robotic controlled cold atmospheric plasma surgery according to claim 1, wherein said step of defining a tissue area to be treated comprises:
detecting an area inputted by a user by manually moving a cold atmospheric probe connected to a robotic end-effector by counting a number of steps of a robotic motor in a direction.

10. A method for robotic controlled cold atmospheric plasma surgery according to claim 1, wherein said step of defining a tissue area to be treated comprises:
inputting boundaries of the tissue area to be treated;
detecting with a camera a projected surface shape of the tissue area to be treated;
calculating with said processor a deformation of the projected surface shape to define a depth map of said tissue area to be treated.
